(19) United States
(12) Patent Application Publication
(10) Pub. No.: US 2003/0077668 A1
(43) Pub. Date: Apr. 24, 2003

(54) Method for arteriosclerosis diagnosis

(75) Inventors: Kazuo Uchida, Kyoto (JP); Shinichi Mashiba, Kyoto (JP)

Correspondence Address: BIRCH STEWART KOLASCH & BIRCH, PO BOX 747, FALLS CHURCH, VA, 22040-0747, US

(73) Assignee: Ikagaku Co., Ltd.

(21) Appl. No.: 10/251,797

(22) Filed: Sep. 23, 2002

Related U.S. Application Data
(63) Continuation-in-part of application No. 09/619,148, filed on Jul. 19, 2000, now abandoned.

(30) Foreign Application Priority Data
Jul. 22, 1999 (JP) 11-207913
Jun. 20, 2000 (JP) 2000-12210

(51) Int. Cl. G01N 33/53; G01N 33/567

(52) U.S. Cl. 435/7.2

(57) ABSTRACT

A novel method for detecting LDL and denatured LDL (particularly, oxidized LDL) having a significant concerning with the onset and progress of arteriosclerosis and Alzheimer's disease is provided, wherein a complex of denatured low density lipoprotein (particularly, oxidized LDL) with an acute phase reactant, blood coagulation.fibrinolytic related protein or disinfectant substance produced by macrophage is used as a measuring subject.

### CROSS-REFERENCE TO RELATED APPLICATION

This is a continuation-in-part application of U.S. patent application Ser. No. 09/619,148 filed Jul. 19, 2000.

### BACKGROUND OF THE INVENTION

1. Field of the Invention

The present invention is based on a finding that oxidized LDL is present in blood, in the form of a complex with various acute phase reactants produced in a process of an acute phase response (a part of acute phase response protein produces also macrophage) in a living body, with various coagulation-fibrinolytic system related proteins, or with various disinfectant substances produced by macrophage and is related to a novel method for arteriosclerosis diagnosis based on the above finding.

2. Description of the Related Art

Arteriosclerosis occurs frequently in the aorta, coronary artery, cerebral artery and carotid artery, and is a disease constituting a main cause of myocardial infarction, cerebral infarction and the like. Recently, Alzheimer's disease also has been found to be a disorder having a significant relation to arteriosclerosis. Conventionally, there is no measuring subject for directly reflecting these conditions of arteriosclerosis in a living body, and such conditions are measured in terms of lipoproteins concerning arteriosclerotic lesion having a strong relation to lipid accumulation on blood vessel wall, mainly composed of LDL such as LDL, LP(a), remnant lipoprotein, Small, dense LDL and the like in serum or plasma. In particular, since indications of a relationship between oxidized LDL and the progress of atherosclerotic lesion made by Steinberg, D. et al., Engl. Med. 320: 915, 1989, and on the other hand, by an injury reaction hypothesis suggested by Ross, R. et al., Nature. 362: 801, 1993, notification has been made of concern towards oxidized LDL in the progress of arteriosclerosis has been notified.

Further, recently, studies considering arteriosclerosis as an inflammation are flourishing (Ross, R. New. Engl. J. Med. 340: 115-126, 1999). According to Junichiro Nishi, et al., Arteriosclerosis, 24: 363-367, 1996 regarding to the relationship between an acute phase response and arteriosclerosis, a living body, in receiving an external stimulus such as infection, injury and the like, quickly performs exclusion of a pathogen and recovery of tissue disorder to maintain homeostasis, through edema caused by pyrexia and sthenia of vaspermeability, homeostasis caused by platelet flocculation and coagulation, and activation of an immunocompetent cell. This vital reaction is called an acute phase response. This vital reaction against an external stimulus such as infection, injury and the like is an important defense mechanism developed in the evolution of mankind. On the other hand, drastic changes in the life environments of the human race, has reduced this external stimulus, whereas, a result generated was of an increase also in an in vivo modulator such as oxidized LDL, advanced glycation endoproduct (AGE) and the like, through changes in the internal environment such as hyperlipidemia, hyperglycemia and the like, since the beginning of this century. On macrophage cells and vascular endothelial cells, specific receptors recognizing these in vivo modulators are generated, and a living body has a mechanism to treat the in vivo modulators via these cells. This means that oxidized LDL and AGE act as foreign materials in a body, and activation of macrophage and endothelial cells are unexpectedly caused in this “foreign material treating process”. Namely, this process composed of a series of steps can be regarded as a process for forming arteriosclerosis.

In the above-mentioned vital reaction, namely, in the process of an acute phase response, a substance significantly increasing in plasma is called an acute phase reactant or acute phase response protein, and main examples thereof include substances as shown in Table 1 (Steel D M, et al., Immuol Today, 15: 81-88, 1994).

TABLE 1
Kind off typical acute phase response protein

| Major APRs | Complement proteins | Coagulation proteins |
|---|---|---|
| C-reactive protein | C2, C3, C4, C5, C9 | Fibrinogen |
| Serum amyloid A | Factor B | von willebrand factor |
| Serum amyloid P | C1 inhibitor | |
| component | C4 binding protein | |
| Proteinase inhibitors | Metal-binding proteins | other proteins |
| α1-antitrypsin | Haptoglobin | α1-add glycoprotein |
| α1-antichymotrypsin | Hemopexin | Heme oxygenase |
| α2-antiplasmin | Ceruloplasmin | Mannose-binding protein |
| Heparin cofactar II | Manganese superoxide | Lipoprotein (a) |
| Plasminogen activator | dismutase | LPS binding protein |
| inhibitor 1 | | Fibrronectin |
| α2-macroglobrin | | |

*A part of data of Steel MD et al. is modified (Steel DM.et al. Immunol Today. 15:81, 1994)

On the other hand, localization of an acute phase reactant at a human aorta atherosclerotic lesion has been confirmed by an immunohistochemical stain method and in situ hybridization method. Descriptions regarding CRP, SAA, SAP are found in Kaoru Hatanama, et al., Arteriosclerosis, 24: 551-555, 1997, descriptions regarding fibrinogen and decomposed products thereof are found in Bini, A. et al., Arteriosclerosis, 9: 109-121. 1989, and descriptions regarding α1-antitrypsin are found in Motohiro Takeya, unpublished literature, 1999.

Under the present conditions, shared roles of various acute phase reactants in arteriosclerosis contains a lot of unclear points, and the progress of studies in the future is expected. Further, it is known that plasminogen activator inhibitor (PAI) activity and thrombin receptor which are inhibiting factors of a tissue plasminogen activator (t-PA) concerning lysis of fibrin clot formed in a blood vessel are also accentuated simultaneously, together with excess expression of tissue factors (TF), in arteriosclerotic lesion, and these factors also participate in accentuation of coagulation of arteriosclerotic intima (Hisao Ogawa, Saishin Igaku, 54: 1210-1217, 1999). Moreover, it isalso known that various pathologic phenomena occurring on artery walls along with the generation and progress of arteriosclerosis are developed by the complicated mutual action of various factors of the coagulation fibrinolytic system, namely, a part of arteriosclerotic lesion tends to become “a site” of clot formation (Kenzo Tanaka, Nippon Ronen Igakukai Zasshi, 35: 880-890, 1998). Further, in the case of atherosclerosis, when LDL in blood is deposited on the wall of avessel, an endothelial cell is activated and a monocyte in blood invades therein and becomes macrophage, and LDL deposited on the wall of a vessel is treated as a foreign material, as described above, and additionally, it is also known, according to recent reports, that the infection of Chlamydia pneumoniae and Helicobactor pylorie is connected with the onset and progress of arteriosclerosis (Murat V, et al., JID. 177: 725-729, 1998) (Patel P, et al. BMJ. 311: 711-714, 1995), and further, the presence of these pathogenic microbes at arteriosclerotic lesions has been confirmed. Therefore, macrophage invaded into arteriosclerotic lesions are judged to have an ability to release various disinfectant substances at the wall of a vessel also for excluding these pathogenic microbes, in addition to the treatment of LDL deposited on the wall of a vessel.

On the other hand, in contrast with the progress of resolution of factors regarding the onset and progress of arteriosclerosis, there has been no method for measuring denatured LDL (oxidatively denatured LDL) in blood readily and correctly. Consequently, an aim of the present study is to provide a novel method for detecting oxidized LDL which has a significant relationship to the onset and progress of arteriosclerosis and Alzheimer's disease.

### SUMMARY OF THE INVENTION

As the main constituent component of a living body, protein, lipid, saccharide and nucleic acid are listed, and lipid is the most easily oxidized, and causes an oxygen addition reaction to form what is called lipid peroxide. The reason for the ease with which lipid is oxidized is that a lot of lipids are in the form of esters of highly unsaturated fatty acids such as linolic acid and arachidonic acid. Lipoprotein is constituted of lipid and protein, and when lipoprotein is oxidized, both the lipid and protein are denatured by oxidization.

As a trigger to oxidize nonenzymatically this bio-lipid, an active oxygen is envisaged. For measuring this lipid peroxide, an analytical chemical means such as normal phase HPLC and the like is used, and it has been proved that nonenzymatic oxidation of lipid securely occurs also in a healthy body (Yoshihiro Yamamoto, et al., Protein.nucleic acid.Enzyme 44: 1253, 1999).

Under the present conditions, as described above, though the prehension of the total amount of lipid peroxide in blood is possible, a method for finding the degree of oxidative denaturing of each lipoprotein does not now exist, the proof for presence of an oxidative denatured body of LDL in blood has been accomplished for the first time by a method according to Japanese Patent Application No. 317162 of 1996 of the present inventors. Further, the present inventors have also found that the detection of oxidatively denatured LDL in blood is possible also by means disclosed in Japanese Patent Application Nos. 109001 of 1999 and 207913 of 1999.

As a result of further repeated studies and investigations thereafter, a fact has been found that oxidatively denatured LDL is present in circulating blood, in the form of a complex with various acute phase reactants, with blood coagulation.fibrinolytic system related protein, or with various disinfectant substances produced by macrophage (Tatsuo Taki, et al., Igaku no Ayumi, 156: 194-197, 1991), leading to the completion of the present invention. Namely, a method for detecting oxidized LDL in blood has been established by expanding the means according to Japanese Patent Application Nos. 317162 of 1996 and 207913 of 1999, completing the present invention.

More specifically, the present inventors have found that when LDL is oxidatively denatured under a blood vessel inner wall, for example, oxidized LDL is present in blood forming a complex with a typical acute phase response protein as shown in Table 1 (α1-antitrypsin, fibrinogen, fibronectin, CRP, SAA, SPA, α1-antichymotrypsin, α1-acidoglycoprotein, α2-macroglobulin) which has invaded into the focus from vessel cavity or has been produced by macrophage, further that oxidized LDL is present in blood forming a complex also with a blood coagulation fibrinolytic protein (tissue factor, plasminogen, prothrombin, thrombin, antithrombin 3, plasmin activator inhibitor 1 and the like) participating in the accentuation of flocculation on an arteriosclerotic inner film, and oxidized LDL is present in blood forming a complex also with a disinfectant substance such as myeloperoxidase, lactoferrin, lysozyme, basic protein and the like released in a foreign material treating process by macrophage that have invaded an arteriosclerotic lesion. In this specification, the above acute phase response protein, blood coagulation-fibrinolytic protein and disinfectant substance are referred to as “marker protein” as the case may be.

It has also been found that when the above complex concentrations of arteriosclerosis patients and healthy persons are compared, the former group shows significantly higher values than the latter.

As described above, the present invention has been achieved on the basis of the discovery that the foregoing complex exists in human blood, not in an arteriosclerosis patient's lesion and that its concentration has a close relationship with the onset and progress of arteriosclerosis.

Typically, the present invention provides a method in which a specific antibody to various antigens forming a complex with oxidized LDL is produced in the same manner as in the production of an antibody recognizing specifically a complex of oxidized LDL with α1-antitrypsin (Japanese Patent Application No. 317162 of 1996), or a complex of oxidized LDL with various proteins is reacted using an antihuman fibrinogen antibody (manufactured by DAKO) as a solid phase antibody, then, an antihuman ApoB antibody labeled with a marker typically including en enzyme is reacted, to detect oxidatively denatured LDL in blood.

The method for arteriosclerosis diagnosis according to the present invention is characterized as follows: blood drawn from a patient or an LDL fraction prepared from this blood is used as a sample, a concentration of a complex present in the sample comprising oxidized LDL and a marker protein selected from the group consisting of an acute phase reactant protein, a blood coagulation-fibrinolytic related protein and a disinfectant substance produced by macrophages is quantitatively measured by an immunological measuring means, and whether the concentration thus measured is significantly higher than that of a group of healthy persons is determined so as to diagnose the onset and progress of arteriosclerosis.

What can be exemplified as an acute phase reactant protein is a protein selected from the group consisting of α1-antitrypsin, fibrinogen, fibronectin, lipoprotein (a), C-reactive protein (CRP), serum amyloid A (SAA), serum amylod P component (SAP), α2-macroglobulin, α1-antichymotrypsin, α1-acidoglycoportein and a complement component.

As an example of a blood coagulation-fibrinolytic related protein, a protein selected from the group consisting of a tissue factor, plasminogen, prothrombin, thrombin, antithrombin 3 and a plasmin activator inhibitor 1 can be given.

An example of a disinfectant substance produced by macrophages is a protein selected from the group consisting of myeloperoxidase, lactroferrin, lysozyme and basic protein.

In the present invention, serum or plasma obtained from a patient's blood can be used as a sample, but to prevent deteriorated measuring accuracy a preferable sample is an LDL fraction obtained from a patient's blood (serum or plasma) by an ultra-centrifugal method or by reacting a dextran sulfate reagent. The arteriosclerosis diagnosis according to the present invention is conducted by measuring quantitatively by an immunological measuring means a concentration of a complex of oxidized LDL and a marker protein present in the above sample.

As an immunological measuring means, a known means, such as an enzyme immunoassay, a latex flocculation method, an immunological emission spectrochemical analysis and an immunochromato method, can be used in the present invention. Particularly preferable is an enzyme immunoassay because it has superior measuring sensitivity and easily provides a kit.

Besides, the present invention is based on the concept that an antigen to be measured is a complex of oxidized LDL and a marker protein present in a sample. As an antibody to detect such an antigen, therefore, it is preferable to use one or both of an antibody for detecting a marker protein forming a complex with oxidized LDL or an antibody for detecting apoB100 present in oxidized LDL forming a complex with this marker protein. When both antibodies are used as immunological measuring means, it is preferable to make the former a primary antibody and the latter a secondary antibody.

As a primary antibody, a monoclonal antibody specifically recognizing a native type marker protein or a polyclonal antibody can be used. It is confirmed by the present inventors that a marker protein forming a complex with oxidized LDL does not always have a native amino sequence and that it is occasionally present partially degraded. But having the property of binding by antigen-antibody reaction to a monoclonal antibody specifically recognizing a native marker protein or a polyclonal antibody, a marker protein forming a complex with oxidized LDL is a preferable primary antibody.

To enhance reaction specificity, a monoclonal antibody prepared with a complex of oxidized LDL and a marker protein as an antigen is a preferable primary antibody. Specifically, such a monoclonal antibody can be prepared as follows: a particular purified marker protein is added to LDL prepared by purifying human serum, a solution of copper sulfate is added to the mixture, and the subsequent mixture is left overnight at 37° C. Using as an antigen an oxidized LDL/marker protein complex thus obtained, a monoclonal antibody can be prepared by a known means. It is confirmed that an antibody thus obtained does not react to a native marker protein, reacting only to a marker protein forming a complex with oxidized LDL, and has very high antigen specificity.

As a secondary antibody, a monoclonal antibody specifically recognizing native type apoB100 or a polyclonal antibody can be used. It is confirmed by the present inventors that apoB100 present in oxidized LDL forming a complex with a marker protein has no native amino sequence and is present partially degraded. But because the foregoing apoB100 has the property of being bound by antigen-antibody reaction to a monoclonal antibody specifically recognizing native apoB100 or a polyclonal antibody, such a monoclonal or polyclonal antibody is a preferable secondary antibody.

When an enzyme immunoassay is used as an immunological measuring means, labeled enzyme can be used, bound to the above secondary antibody by a known method.

To enhance reaction specificity, a monoclonal antibody prepared with oxidized LDL as an antigen is preferred as a secondary antibody. To be specific, a solution of copper sulfate is added to LDL prepared from human serum, and the mixture is left overnight at 37° C. A monoclonal antibody can be prepared by a known method with thus artificially prepared oxidized LDL as an antigen. An antibody thus obtained has high antigen specificity to ApoB100 present in oxidized LDL forming a complex with a marker protein.

When an enzyme immunoassay is used as an immunological measuring means, labeled enzyme can be used, bound with the above secondary antibody by a known method.

The following are preferred examples of using a polyclonal antibody as a primary antibody. First, when an acute phase reactant forming a complex with oxidized LDL is used as an antigen, a preferable primary antibody is a polyclonal antibody selected from the antihuman polyclonal antibody group consisting of α1-antitrypsin, fibrinogen, fibronectin, lipoprotein (a), C-reactive protein (CRP), serum amyloid A (SAA), serum amylod P component (SAP), α2-macroglobulin, α1-antichymotrypsin, α1-acidoglycoportein and a complement component.

When a blood coagulation-fibrinolytic related protein forming a complex with oxidized LDL is used as an antigen, a preferable primary antibody is a polyclonal antibody selected from the antihuman polyclonal antibody group consisting of a tissue factor, plasminogen, prothrombin, thrombin, antithrombin 3 and a plasmin activator inhibitor 1.

When a disinfectant substance produced by macrophages, forming a complex with oxidized LDL, is used as an antigen, a preferable primary antibody is a polyclonal antibody selected from the anti-human polyclonal antibody group consisting of myeloperoxidase, lactroferrin, lysozyme and basic protein.

An immunological measuring means used in the present invention is not limited to any particular method as mentioned above. Described below as a specific example is an oxidized LDL/marker protein complex detecting scheme in which an LDL fraction obtained from human serum is sampled and the sandwich ELISA method using the above primary and secondary antibodies is adopted (no washing after each reaction included). This oxidized LDL/marker protein complex detecting method is characterized in that after an oxidized LDL/marker protein complex present in an LDL fraction is bound to a primary antibody fixed to a carrier, a secondary antibody bound to labeled enzyme is bound to the foregoing oxidized LDL/marker protein complex, and an oxidized LDL/marker protein complex in the sample is quantitatively measured by enzyme-stroma reaction. Providing high detecting sensitivity and fast detecting speed, namely, only a few hours, the sandwich ELISA method is preferable for use in the present invention.

When a secondary antibody bound to labeled enzyme is used as above, data obtained only show absorbance, and no complex concentration is available. So an artificially prepared oxidized LDL/marker protein complex is used, and a calibration curve is drawn plotting absorbance degrees corresponding to complex concentrations. Thus, a concentration of an oxidized LDL/marker protein complex present in a sample can be quantitatively measured.

In the present invention, the onset and progress of arteriosclerosis is diagnosed by determining whether a concentration of an oxidized LDL/marker protein complex obtained from a patient is significantly higher than that previously obtained from a healthy person. It is confirmed by the present inventors that the above complex concentrations of arteriosclerosis, diabetes and multiple risk factor syndrome patients statistically show significantly higher values than those of healthy people. So if the complex concentrations of many healthy people are measured and their distributions are statistically shown, it can be judged whether the concentration of a particular patient is significantly higher, and thus whether the patient has contracted arteriosclerosis or how far it has progressed.

In determining significant differences between the foregoing complex concentrations, either the parametric or the non-parametric method can be used. But generally, since the complex concentrations are not normally distributed, the non-parametric method is preferred.

To conduct the arteriosclerosis diagnosis according to the present invention efficiently, it is preferable to use a diagnostic kit provided at least with the foregoing immunological measuring means. Specifically, a known method, such as an enzyme immunoassay, a latex flocculation method, an immunological emission spectrochemical analysis and an immunochromato method, can be used. In the present invention, an enzyme immunoassay is most preferred because it has superior measuring sensitivity and easily provides a kit.

The present invention is based on the concept that an antigen to be measured is a complex of oxidized LDL and a marker protein present in a sample. As an antibody to detect such an antigen, therefore, it is preferable to use one or both of an antibody for detecting a marker protein forming a complex with oxidized LDL or an antibody for detecting apoB100 present in oxidized LDL forming a complex with this marker protein. In the present invention, it is preferable to provide a diagnostic kit with such an antibody used as a reagent or fixed to a carrier like a microplate.

If the sandwich ELISA method is used as an immunological measuring means in the present invention, it is preferable as mentioned above to use as a primary antibody an antibody for detecting a marker protein forming a complex with oxidized LDL and as a secondary antibody an antibody for detecting apoB100 present in oxidized LDL forming a complex with this marker protein. In the present invention, it is preferred to provide a diagnostic kit with the above primary antibody fixed to a carrier like a microplate and with the above secondary antibody used as a reagent.

In addition to the foregoing immunological measuring means, a diagnostic kit for the present invention may be provided with an oxidized LDL/marker protein complex artificially prepared for calibration curve drawing. This saves the trouble of preparing a separate oxidized LDL/marker protein complex and makes it possible to easily draw a calibration curve and to quantitatively measure a concentration of an oxidized LDL/marker protein complex present in a sample.

In addition to the foregoing immunological measuring means and reagent for calibration curve drawing, a diagnostic kit for the present invention may be provided with a dextran sulfate reagent. This makes it possible to easily divide blood drawn from a patient into LDL fractions and to simplify a series of operations.

### BRIEF DESCRIPTION OF THE SEVERAL VIEWS OF THE DRAWINGS

FIG. 1 shows graphs comparing the concentrations of complexes of LDL or denatured LDL with acute phase response protein (A), coagulation.fibrinolytic system protein (B) and disinfectant protein (C), among three groups with different blood lipid concentration.

FIG. 2 is a graph showing an LDL-fibrinogen related substance complex, an LDL-fibronectin complex and a collagen bonding lipoprotein, present in an LDL fraction.

FIG. 3 provides graphs showing a relationship between the blood Lp(a) concentration and extracellular substrate protein (collagen) bonding Lp(a) concentration, a relationship between the blood LDL-cholesterol concentration and the concentration of novel lipoprotein concerning arteriosclerotic lesion, and a relationship between the concentration of an LDL-fibrinogen related substance complex and the concentration of collagen bonding LDL.

FIG. 4 is a graph showing the distribution of the concentration of an LDL-fibrinogen related substance complex in serum of a healthy person.

FIG. 5 is a graph showing comparison of the amounts of an LDL-fibrinogen related substance complex in serum of a healthy person, a diabetic mellitus patient and a multiple risk factor syndrome patient.

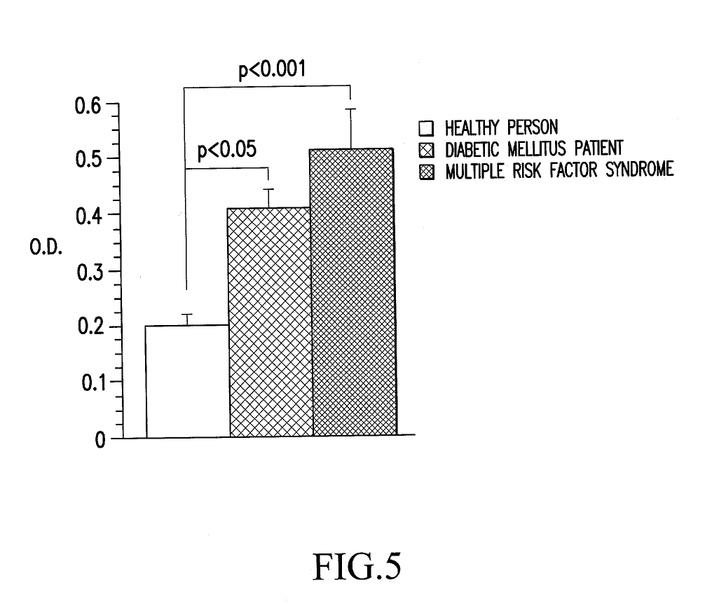

FIG. 6 is a graph showing distribution of concentrations of AT/OxLDL, fib/OxLDL, SAA/OxLDL, CRP/OxLDL complexes in the serums of a group of healthy persons (those taking health examinations) and a group of coronary artery disease patients (those found by photograph examination with more than 50% stricture in their main coronary arteries).

FIG. 7 are graphs for review of the formation mechanism of oxidized LDL-(serum amyloid A1; SAA1) complex.

### DETAILED DESCRIPTION OF THE PREFERRED EMBODIMENTS

A. Example for Detecting Oxidized LDL Forming Complex with Acute Phase Reactant

(A-1) [Measurement of Oxidized LDL/CRP Complex in LDL Prepared by an Ultra-Centrifugal Method or Dextran Sulfate/Ca Precipitation Method]

1. An antihuman CRP polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized/CRP complex in a sample is calculated based on a calibration curve obtained by using an oxidized LDL/CRP complex prepared artificially.

(A-2) [Measurement of Oxidized LDL/Amyloid A Complex in LDL Prepared by an Ultra-Centrifugal Method or Dextran Sulfate/Ca Precipitation Method]

1. An antihuman amyloid polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/amyloid A complex in a sample is calculated based on a calibration curve obtained by using an oxidized LDL/amyloid A complex prepared artificially.

(A-3) [Measurement of Oxidized LDL/α2-Macroglobulin Complex in LDL Prepared by an Ultra-Centrifugal Method or Dextran Sulfate/Ca Precipitation Method]

1. An antihuman α2-macroglobulin polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 2501 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/α2-macroglobulin complex in a sample is calculated based on a calibration curve obtained by using an oxidized LDL/α2-macroglobulin complex prepared artificially.

(A-4) [Measurement of an Oxidized LDL/α1-Antichymotrypsin Complex in LDL Prepared by an Ultra-Centrifugal Method or Dextran Sulfate/Ca Precipitation Method]

1. An antihuman α1-antichymotrypsin polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/α1-antichymotrypsin complex in a sample is calculated based on a calibration curve obtained by using an oxidized LDL/α1-antichymotrypsin complex prepared artificially.

(A-5) [Measurement of an Oxidized LDL/α1-Acidoglycoprotein Complex in LDL Prepared by an Ultra-Centrifugal Method or Dextran Sulfate/Ca Precipitation Method]

1. An antihuman α1-acidoglycoprotein polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/α1-acidoglycoprotein complex in a sample is calculated based on a calibration curve obtained by using an oxidized LDL/α1-acidoglycoprotein complex prepared artificially.

B. Example for Detecting Oxidized LDL Forming Complex with Blood Coagulation.Fibrinolytic System Related Protein

(B-1) [Measurement of Oxidized LDL/Thrombin Complex in LDL Prepared by an Ultra-Centrifugal Method or Dextran Sulfate/Ca Precipitation Method]

1. An antihuman thrombin polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/thrombin complex in a sample is calculated based on a calibration curve obtained by using an oxidized LDL/thrombin complex prepared artificially.

(B-2) [Measurement of an Oxidized LDL/Antithrombin 3 Complex in LDL Prepared by an Ultra-Centrifugal Method or Dextran Sulfate/Ca Precipitation Method]

1. An antihuman antithrombin 3 polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250>1/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/antithrombin 3 complex in a sample is calculated based on a calibration curve obtained by using an oxidized LDL/antithrombin 3 complex prepared artificially.

(B-3) [Measurement of an Oxidized LDL/Plasminogen Activator Inhibitor 1 Complex in LDL Prepared by an Ultra-Centrifugal Method or Dextran Sulfate/Ca Precipitation Method]

1. An antihuman plasminogen activator inhibitor 1 polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/plasminogen activator inhibitor 1 complex in a sample is calculated based on a calibration curve obtained by using an oxidized LDL)/plasminogen activator inhibitor 1 complex prepared artificially.

C. Example for Detecting Oxidized LDL Forming a Complex with a Disinfectant Substance Produced by Macrophage

(C-1) [Measurement of an Oxidized LDL/Myeloperoxidase Complex in LDL Prepared by an Ultra-Centrifugal Method or Dextran Sulfate/Ca Precipitation Method]

1. An antihuman myeloperoxidase polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 250 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 250 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/myeloperoxidase complex in a sample is calculated based on a calibration curve obtained by using an oxidized LDL/myeloperoxidase complex prepared artificially.

(C-2) [Measurement of an Oxidized LDL/Lactoferrin Complex in LDL Prepared by an Ultra-Centrifugal Method or Dextran Sulfate/Ca Precipitation Method]

1. An antihuman lactoferrin polyclonal antibody (manufactured by DAKO) is added at a ratio of 10 μg/ml to a 0.05M Tris-HCl (containing 0.15M NaCl, pH 8.0) buffer solution, and poured onto a microplate at a ratio of 100 μl/well.

2. After physical adsorption over night at 4° C., the microplate in use is washed three times with de-ionized water, and a 0.05M Tris-HCl buffer solution (pH 7.5) containing 0.1% sucrose, bovine serum albumin and 0.05% sodium azide is poured onto a microplate at a ratio of 10 μl/well, left still for 30 minutes or more at room temperature, then, the solution is discarded, and the plate is dried at 4° C. The dried microplate is washed three times with de-ionized water at a ratio of 2501 μl/well.

3. A 1% bovine albumin solution containing 55 mg/ml Mouse Gamma Globulin and Goat Gamma Globulin is poured onto a microplate at a ratio of 100 μl/well, and to this is added 50 μl of a sample or standardized solution.

4. Reaction for 1.5 hours at 37° C.

5. Washing five times with a 0.005% Tween 20 solution at a ratio of 250 μl/well.

6. A solution of a biotin labeled Fab′-nized IgG-apoB/427 monoclonal antibody controlled to 1.6 μg/ml with a 1% BSA solution is poured at a ratio of 100 μl/well.

7. Reaction for 1.5 hours at 37° C.

8. Washing five times with a 0.005% Tween 20 solution at a ratio of 2501 μl/well, as in 3.

9. HRP labeled avidin D (manufactured by Vector laboratories) is diluted 15000-fold with a 1% casein solution, and the resulted solution is poured at a ratio of 100 μl/well.

10. Reaction for 30 minutes at 37° C.

11. Washing five times with a 0.005% Tween 20 solution at a ratio of 250 μl/well.

12. A coloration reagent composed of a hydrogen peroxide solution and a TMBZ solution is poured at a ratio of 100 μl/well, and reacted for 30 minutes at room temperature.

13. A 1M phosphoric acid aqueous solution is poured at a ratio of 100 μl/well to terminate the reaction.

14. Photometry at a main wavelength of 450 nm and a sub wavelength of 630 nm.

15. The concentration of an oxidized LDL/lactoferrin complex in a sample is calculated based on a calibration curve obtained by using an oxidized LDL/lactoferrin complex prepared artificially.

D. Comparison of Concentration of Complex of LDL or Denatured LDL (Oxidized LDL) with Acute Phase Response Protein, Coagulation-Fibrinolytic Protein or Disinfectant Protein, in Serum Containing Lipid in Various Concentrations.

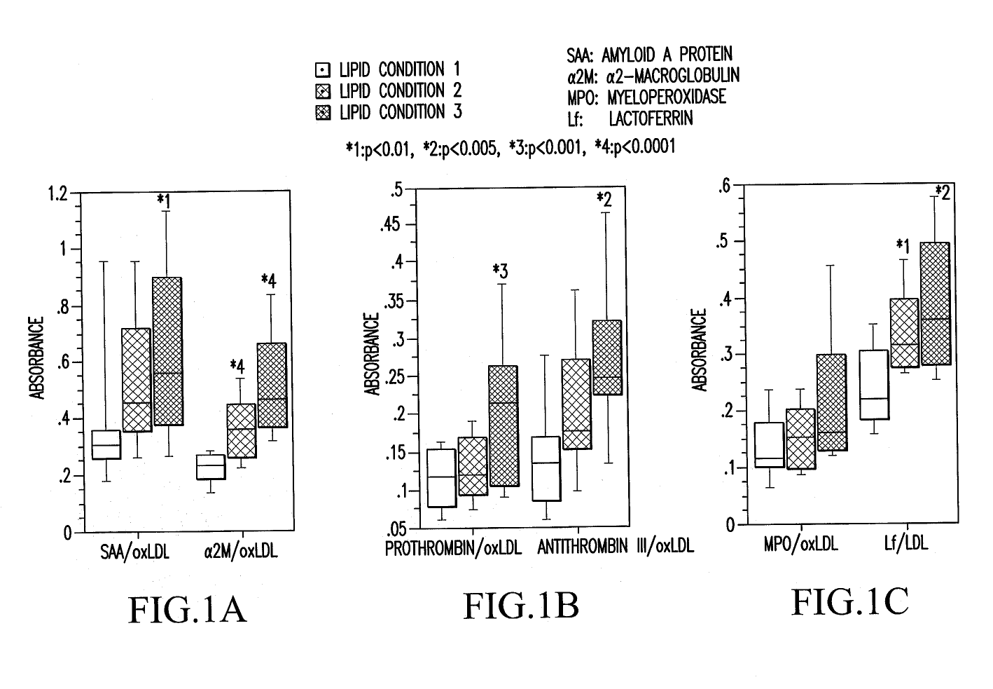

Regarding three groups, a first group under a lipid condition in serum (cholesterol 160 mg/dl>, neutral lipid 10 mg/dl>, HDL cholesterol 40-90 mg/dl), a second group under a lipid condition in serum (cholesterol 161-219 mg/dl>, neutral lipid 101-139 mg/dl>, HDL cholesterol 40-90 mg/dl) and a third group under a lipid condition in serum (cholesterol 220 mg/dl<, neutral lipid 140 mg/dl<, HDL cholesterol 40 mg/dl>), the serum concentrations of (amyloid A protein and α2-macroglobulin) as examples of measuring a complex of LDL or denatured LDL (oxidized LDL) with an acute phase response protein, of (prothrombin and antithrombin 3) as examples of measuring a complex of LDL or denatured LDL (oxidatively denatured) with an aggregation.fibrinolytic system protein and of (myeloperoxidase and lactoferrin) as examples of measuring a complex of a disinfectant substance produced by macrophage with LDL or denatured LDL (oxidized LDL) were measured, to find the highest concentration in the third group (hyperlipidemia group) in any of the complexes (see FIG. 1).

E. Prior Application (Japanese Patent Application No. 207913 of 1999)

(E-1) History Until Prior Application

Previous to the prior application, the present inventors had tried formation of a complex of LDL with fibrinogen or LDL with fibronectin, and confirmed that a complex is formed by LDL received artificial oxidation denaturing. Namely, crude fibrinogen or fibronectin was added to native LDL, saccharified LDL and oxidized LDL, and an investigation was conducted to determine which LDL form a complex with the fibrinogen or fibronectin. Mixed samples of respective LDLs with the fibrinogen or fibronectin were subjected to electrophoresis, then, the lipids were stained with Fat red 7B and the fibrinogen or fibronectin was stained by an immunoblot method.

As a result, no complex was formed by the native LDL and by the mixed sample of the saccharified LDL with the fibrinogen or fibronectin, whereas, the mixed sample of the oxidized LDL with the fibrinogen or fibronectin prepared by vascular endothelial cell treatment or copper sulfate treatment formed a complex (oxidized LDL-fibrinogen complex or oxidized LDL-fibronectin complex). Further, sera of diabetes mellitus and myocardial infarction patients were separated by ultra-centrifugation to obtain LDL (1.006 g/ml<d<1.063 g/ml), which was subjected to an antihuman fibronectin immunoaffinity chromatograph means to purify and isolate an LDL-fibrinogen complex and an LDL-fibronectin complex. As for the natures of LDL forming these LDL-fibrinogen complex and LDL-fibronectin complex, increase of a lipid peroxide characteristic to oxidized LDL, decomposition of ApoB protein and increase of negative charge of the whole LDL particle were observed. Further, from ELISA using fractions obtained by gel permeation analysis, the presences of an LDL-fibrinogen complex and an LDL-fibronectin complex were recognized in the LDL fraction.

Therefore, the present inventors noticed a fact that LDL or oxidized LDL is present carrying such a convenient label as fibronectin and supposed that if a monoclonal antibody, specifically recognizing the fibronectin forming a complex with oxidized LDL, can be produced, recognition, measurement and purification and isolation of a complex of LDL or oxidized LDL in blood with fibronectin can be effected using this antibody.

As the antigen in producing the antibody, an artificially prepared oxidized LDL-fibronectin complex was used. The reaction specificity of the resulted antigen against fibronectin manifested no reactivity with native fibronectin, however, manifested recognition of fibronectin forming a complex with oxidized LDL. Further, the antibody of the present invention was also judged to give no recognition of ApoB protein.

(E-2) Method for Producing Antihuman Oxidized LDL Bonding Fibronectin Monoclonal Antibody

(One Example of Method for Producing Monoclonal Antibody)

[Preparation of Antigen]

LDL (1.006 g/ml<d<1.063 g/ml) obtained from human serum by ultra-centrifugal separation was passed through an immunoaffinity column using an antihuman α1 antitrypsin polyclonal antibody to remove an LDL-α1 antitrypsin complex. Purified human fibronectin was added to this LDL free of α1 antitrypsin, to this was added a copper sulfate solution, and the solution was left over night at 37° C. to form an oxidized LDL-fibronectin complex.

The formation of a complex of LDL with fibronectin can be confirmed by performing ELISA (an antihuman fibronectin antibody is used as a solid phase antibody, and an antihuman ApoB antibody is used as an enzyme labeled antibody) on each fraction obtained by gel permeation analysis using the complex as a sample.

[Immunity Against Animals]

This complex (antigen) was controlled using a phosphate buffered physiological saline to be a 1 mg/ml solution in terms of the concentration of protein, and this solution was mixed with the same amount of Freund's adjuvant to obtain an emulsion, which was administered in an amount of 500 μl intraperitoneally to a 6 week old mouse (Balb/C type mouse). This was conducted every two weeks for three times in total.

[Cell Fusion]

Four days after the final immune operation, a spleen lymphocyte collected from the spleen of this mouse was fused with a mouse myeloma cell (P3-X63-Ag8-U1).

The fusion was conducted, using a 50% polyethylene glycol 4000 solution as a fusion accelerating agent, at 37° C. for a fusion time of 10 minutes including operations of addition, mixing and dilution of the fusion accelerating agent, according to an ordinary method. Then, a HAT medium (RPMI medium containing hypoxanthine, thymidine and 10% fetal bovine serum) was poured onto each well, and after 2 to 3 days, the selection of an antibody producing hybridoma was conducted.

For this selection, onto a 96-well microplate on which an oxidized LDL-IgA complex, native IgA and native apoB had been fixed, the culture supernatant of a hybridoma formation colony of each well was poured in an amount of 100 μl, washed, then, 100 μl of a peroxidase labeled anti-mouse immunoglobulin antibody was added to cause an antigen-antibody reaction, washed, and coloration and ELISA were conducted according to ordinary methods, to select a plurality of intended antibody (antibody which shows reactivity with oxidized LDL bonding fibronectin but does not react with native fibronectin and native apoB) producing hybridomas. Then, colonies showing intended antibody production were collected, and cloning was so conducted as to obtain a single colony of a hybridoma by a limiting dilution method. In this method, collected colonies were diluted with a HT medium, and sprayed together with feeder cells so that not more than one hybridoma was contained in each well of a 96-well microplate. The above-described procedure was repeated twice, to obtain a plurality of monocloned antihuman oxidized LDL bonding fibronectin antibody producing hybridomas.

[Peritoneal-Fluidization of Antihuman Oxidized LDL Bonding Fibronectin Monoclonal Antibody]

Pristane (immunosuppressant agent) was administered intraperitoneally to a 8-week old mouse (Balb/C type mouse). Three to seven days later, the antibody producing a hybridoma was administered intraperitoneally, and after 7 days, peritoneal-fluidized antibodies were collected from the abdominal cavity of the mouse.

[Purification of Antibody]

Each antibody obtained following peritoneal-fluidization was salted out twice with 50% ammonium sulfate, purified via dialysis with a phosphate buffered physiological saline, a plurality of antihuman oxidized LDL bonding fibronectin monoclonal antibodies and artificially prepared oxidized LDL-fibronectin complexes were allowed to react respectively, and antihuman oxidized LDL bonding fibronectin monoclonal antibodies (named OFN-1) having excellent sensitivity in ELISA using an antihuman ApoB enzyme labeled antibody as the secondary antibody were selected.

(E-3) According to the present invention, by allowing blood components of a subject to contact with the antibody of the present invention and quantifying the amount of antibodies reacted specifically with the aforementioned antibody, LDL or oxidized LDL-fibronectin complex contained in blood can be measured. This measurement is conducted by a known method such as radio immunoassay, enzyme immunoassay, immunoblot method, immunoprecipitation method, fluorescent immunoassay, chemoluminescent or bioluminescent immunoassay and the like.

The method for measuring LDL or oxidized LDL-fibronectin complex by an enzyme-linked immunosorbent assay (ELISA) will be specifically exemplified below.

[Fixing of Antibody to Microplate]

A 0.1M Tris buffer solution (pH 8.4) containing an antihuman oxidized LID bonding fibronectin monoclonal antibody (OFN-1) (5 μg/ml) is poured in an amount of 100 μl onto each well of a microplate (manufactured by NUNC), and left overnight at 4° C. to allow the antibody to be adsorbed in solid phase.

[Preparation of Enzyme Labeled Antibody]

Separately, an antihuman ApoB polyclonal antibody or an antihuman ApoB monoclonal antibody (manufactured by using oxidized LDL as an antigen) is used giving labeling of Fab′ with pepsin and 2-mercaptoethanolamine, to prepare an enzyme labeled antibody.

[Measurement of Serum or Plasman LDL or Oxidized LDL-Fibronectin Complex]

A Tris buffer solution (0.1M, pH 8.0) containing 1% bovine albumin is poured in an amount of 100 μl onto each well, then, 50 μl of serum or plasma is added and blended into a miscible condition, then, reacted for 2 hours at 37° C. Then, the product is washed three times with a washing solution (phosphate buffer solution containing Tween 20 in the final concentration of 0.005%: 0.02M: pH 7.4).

Thereafter, a peroxidase labeled antihuman ApoB Fab′ antibody solution (Tris buffer solution containing 1% bovine albumin) is added in an amount of 100 μl to each well, then, reacted for 1 hour at 37° C. and washed three times as described above.

For preparing a substrate color developing solution, 1.66 m MTMBZ (manufactured by Doj in Kagaku) is dissolved with methanol, then, a substrate solution obtained by adding a 0.2M Tris buffer solution so as to give a methanol concentration of 50% and a 35 mM citric acid solution containing 0.02% hydrogen peroxide were blended in the same amount to be a miscible condition, 100 μl of the resulted solution is added to each well, left for 10 minutes at room temperature, then, 100 μl of a reaction termination solution (2.5M phosphoric acid solution) is added to each well.

Colors are compared at wavelengths of 450/630 nm using a chromatometer for microplate and the absorbance is calculated. Artificially prepared oxidized LDL-fibronectin complex is allowed to react according to the same procedure as described above, a calibration curve is made, and the concentration of LDL or oxidized LDL-fibronectin complex in the sample is calculated using this calibration curve.

(E-4) Further, according to the present invention, novel lipoprotein concerning arteriosclerotic disease containing LDL or oxidized LDL and fibronectin complex has a stronger tendency to be precipitated onto an extracellular substrate component, therefore, a method for detecting novel lipoprotein concerning arteriosclerotic lesions containing a complex of fibrinogen or fibrin (or decomposed products thereof) with LDL or oxidized LDL connected to an extracellular substrate protein fixed to solid phase and a complex of fibronectin with LDL or oxidized LDL, will be described below. A measuring method using collagen present in almost all tissue of a living body such as skin, bone, tendon, muscle and the like typically including blood vessels, as an extracellular substrate protein for solid phase formation, will be specifically exemplified below.

[Fixing of Extracellular Protein to Microplate]

A 0.1M Tris buffer solution (pH 8.4) containing 10 μg/ml collagen type I is poured in an amount of 100 μl onto each well of a microplate (manufactured by NUNC), and left at 37° C. for evaporation until dry to allow collagen to be adsorbed in solid phase.

[Preparation of Enzyme Labeled Antibody]

Separately, an antihuman ApoB polyclonal antibody or an antihuman ApoB monoclonal antibody (manufactured by using oxidized LDL as an antigen) is used to give a Fab′ with pepsin and 2-mercaptoethanolamine, this Fab′ is labeled with peroxidase to prepare an enzyme labeled antibody.

[Measurement of Serum or Plasma Collagen Bonding Lipoprotein]

A Tris buffer solution (0.1M, pH 8.0) containing 1% bovine albumin is poured in an amount of 100 μl onto each well, then, 50 μl of serum or plasma is added and blended into a miscible condition, then, reacted for 2 hours at 37° C. Then, the product is washed three times with a washing solution (phosphate buffer solution containing Tween 20 in the final concentration of 0.005%: 0.02M: pH 7.4). Thereafter, a peroxidase labeled antihuman ApoB Fab′ antibody solution (Tris buffer solution containing 1% bovine albumin) is added in an amount of 100 μl to each well, then, reacted for 1 hour at 37° C. and washed three times as described above.

For preparing a substrate color developing solution, 1.66 m MTMBZ (manufactured by Doj in Kagaku) is dissolved with methanol, then, a substrate solution obtained by adding a 0.2M Tris buffer solution so as to give a methanol concentration of 50% and a 35 mM citric acid solution containing 0.02% hydrogen peroxide are blended in the same amount to be in a miscible condition, 100 μl of the resulted solution is added to each well, left for 10 minutes at room temperature, then, 100 μl of a reaction termination solution (2.5M phosphoric acid solution) is added to each well.

Colors are compared at wavelengths of 450/630 nm using a chromatometer for microplate and the absorbance is calculated.

Collagen bonding lipoprotein isolated and purified by an affinity column fixing collagen from human serum is allowed to react in the same operation as described above, a calibration curve is made, and the concentration of collagen bonding lipoprotein in the sample is calculated using this calibration curve.

(E-5) Further, according to the present invention, novel lipoprotein concerning arteriosclerotic lesions containing a composite of LDL or oxidized LDL with fibrinogen and fibrin (or decomposed products thereof), or fibronectin has a stronger tendency to be bonded to a polymer compound such as polystyrene, nylon and the like, this lipoprotein can be measured also by a solid phase method. A method using a polystyrene microplate as solid phase will be specifically exemplified.

[Preparation of Enzyme Labeled Antibody]

Separately, an antihuman ApoB polyclonal antibody or an antihuman ApoB monoclonal antibody (manufactured by using oxidized LDL as an antigen) is used to give a Fab′ with pepsin and 2-mercaptoethanolamine, this Fab′ is labeled with peroxidase to prepare an enzyme labeled antibody.

[Measurement of Novel Lipoprotein in Serum or Plasma Concerning Arteriosclerotic Lesions, According to Solid Phase Method]

A Tris buffer solution (0.1M, pH 8.0) containing 1% bovine albumin is poured in an amount of 100 μl onto each well of a non-treated polystyrene microplate (manufactured by NUNC), then, 50 μl of serum or plasma is added and blended into a miscible condition, then, reacted for 2 hours at 37° C. Then, the product is washed three times with a washing solution (phosphate buffer solution containing Tween 20 in the final concentration of 0.005%: 0.02M: pH 7.4).

Thereafter, a peroxidase labeled antihuman ApoB Fab′ antibody solution (Tris buffer solution containing 1% bovine albumin) is added in an amount of 100 μl to each well, then, reacted for 1 hour at 37° C. and washed three times as described above.

For preparing a substrate color developing solution, 1.66 m MTMBZ (manufactured by Dojin Kagaku) is dissolved with methanol, then, a substrate solution obtained by adding a 0.2M Tris buffer solution so as to give a methanol concentration of 50% and a 35 mM citric acid solution containing 0.02% hydrogen peroxide are blended in the same amount to be in a miscible condition, 100 μl of the resulted solution is added to each well, left for 10 minutes at room temperature, then, 100 μl of a reaction termination solution (2.5M phosphoric acid solution) is added to each well. Colors are compared at wavelengths of 450/630 nm using a chromatometer for microplate and the absorbance is calculated. Novel lipoprotein concerning arteriosclerosis containing a complex of LDL or oxidized LDL with fibrinogen and fibrin (or decomposed products thereof) or an LDL or oxidized LDL-fibronectin complex, isolated and purified by an affinity column fixing collagen from human serum, is allowed to react in the same operation as described above, a calibration curve is made, and the concentration of the lipoprotein in the sample is calculated using this calibration curve.

In the present invention, a method for detecting novel lipoprotein concerning arteriosclerosis containing a complex of LDL or oxidized LDL with fibrinogen or fibrin (or decomposed products thereof) will also be specifically exemplified below.

[Fixing of Antibody to Microplate]

A 0.1M Tris buffer solution (pH 8.4) containing an antihuman fibrinogen antibody (manufactured by DAKO) is poured in an amount of 100 μl onto each well of a microplate (manufactured by NUNC), and left overnight at 4° C. to allow the antibody to be adsorbed in solid phase.

[Preparation of Enzyme Labeled Antibody]

Separately, an antihuman ApoB polyclonal antibody or an antihuman ApoB monoclonal antibody is used giving labeling of Fab′ with pepsin and 2-mercaptoethanolamine, to prepare an enzyme labeled antibody.

[Measurement of Serum LDL or Oxidized LDL/Fibrinogen or Fibrin (or Decomposed Products Thereof) Complex (Sometimes may be Abbreviated as LDL-Fibrinogen Related Substance Complex)]

A Tris buffer solution (0.1M, pH 8.0) containing 1% bovine albumin is poured in an amount of 100 μl onto each well, then, 50 μl of serum is added and blended into a miscible condition, then, reacted for 1 hour at 37° C. Then, the product is washed three times with a washing solution (phosphate buffer solution containing Tween 20 in the final concentration of 0.005%: 0.02M: pH 7.4).

Thereafter, a peroxidase labeled antihuman ApoB Fab′ antibody solution (Tris buffer solution containing 1% bovine albumin) is added in an amount of 100 μl to each well, then, reacted for 1 hour at 37° C. and washed three times as described above.

For preparing a substrate color developing solution, 1.66 m MTMBZ (manufactured by Doj in Kagaku) is dissolved with methanol, then, a substrate solution obtained by adding a 0.2M Tris buffer solution so as to give a methanol concentration of 50% and a 35 mM citric acid solution containing 0.02% hydrogen peroxide were blended in the same amount to be in a miscible condition, 100 μl of the resulted solution is added to each well, left for 10 minutes at room temperature, then, 100 μl of a reaction termination solution (2.5M phosphoric acid solution) is added to each well.

Colors are compared at wavelengths of 450/630 nm using a chromatometer for microplate and the absorbance is calculated. Artificially prepared oxidized LDL-fibrinogen complex is allowed to react according to the same procedure as described above, a calibration curve is made, and the concentration of an LDL-fibrinogen related substance complex in the sample is calculated using this calibration curve.

(E-6) Recognition of LDL-Fibrinogen Related Substance Complex, LDL-Fibronectin Complex and Collagen Bonding LDL Present in LDL Fraction

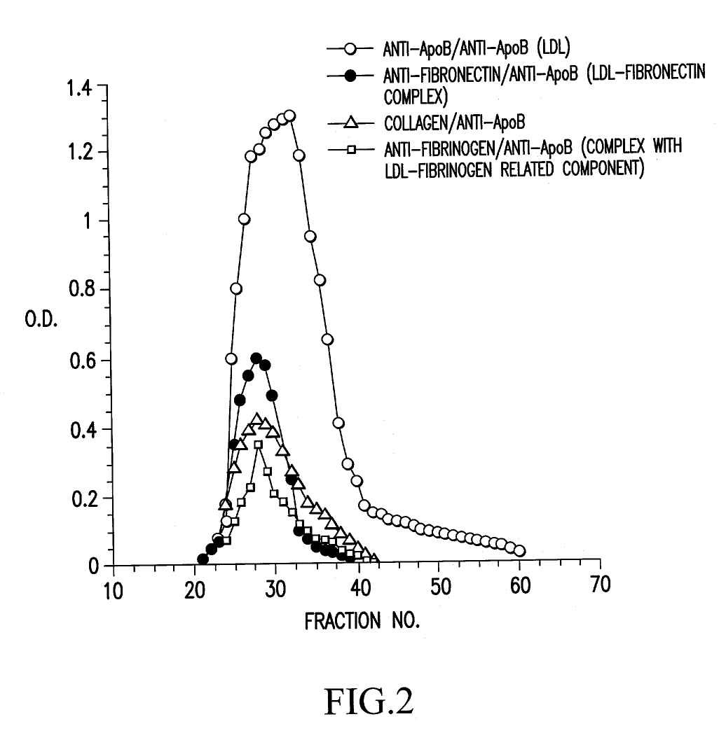

Human serum was ultra-centrifugally separated, and gel permeation analysis was conducted using the resulted LDL (1.006<d<1.063 g/ml) fraction, and ELISA was carried out on each fraction using a combination as described below. Namely, LDL (antihuman ApoB/antihuman ApoB), LDL/fibronectin complex (antihuman fibronectin/antihuman ApoB), LDL/fibrinogen complex (antihuman fibrinogen/antihuman ApoB) and collagen bonding LDL (collagen/antihuman ApoB) were measured, to recognize the presences of an LDL/fibronectin complex, LDL/fibrinogen complex and collagen adhering LDL, in an LDL fraction as shown in FIG. 2.

(E-7) Relationship between Blood Lp(a) Concentration and Collagen Bonding Lp(a) Concentration, and Relationship Between Concentration of Complex of LDL/Fibrinogen Related Substance in Blood and Concentration of Collagen Bonding LDL

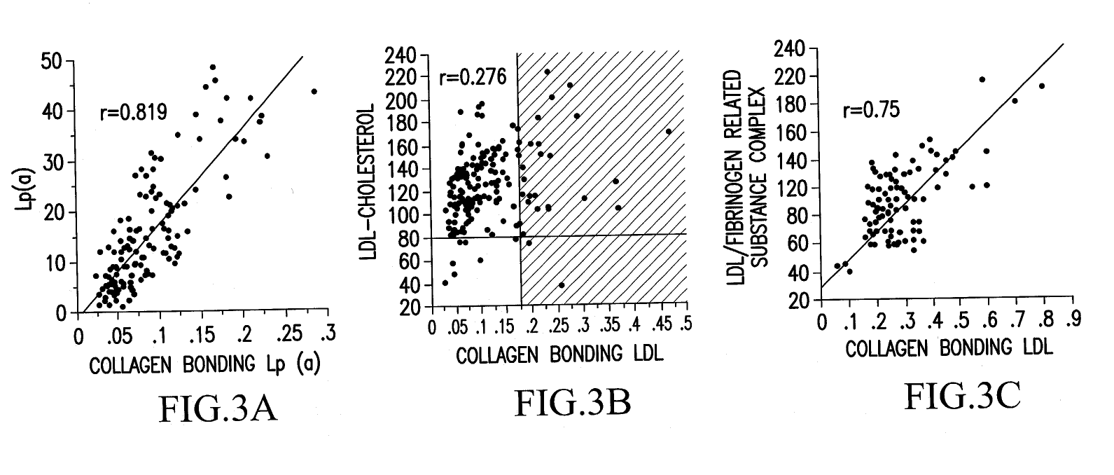

Lp (a) which is regarded as a risk factor in arteriosclerosis due to its bonding property manifested on an extracellular substrate component, is detected as collagen bonding Lp(a) due to blood Lp (a) concentration dependency. Namely, it is indicated that all of Lp(a) present in blood have a bonding property to an extracellular substrate component (FIG. 3a). Cushing et al. suggests a possibility that apoprotein (a) is easily connected with extracellular substrate protein (Arteriosclerosis., 9: 593, 1989). In the case of LDL, only a part thereof shows a bonding property to an extracellular substrate component (FIG. 3b), and the amount of lipoprotein having a bonding property to an extracellular substrate component can not be estimated from the total LDL amount in blood. However, since the correlationship between the concentration of an LDL/fibrinogen complex in blood and the concentration of collagen bonding LDL is excellent as described in FIG. 3c, there is indicated a possibility that the LDL/fibrinogen complex and the collagen bonding LDL are the same substance. Therefore, there is indicated a possibility that the bonding property manifested by LDL to extracellular substrate protein depends on fibrinogen related protein bonded to LDL. Namely, lipoprotein (arteriosclerosis inducing lipoprotein) having the same bonding property to an extracellular substrate component as that of Lp(a) is present in blood LDL.

(E-8) Distribution of Concentration of LDL-Fibrinogen Related Substance Complex in Serum of a Healthy Person

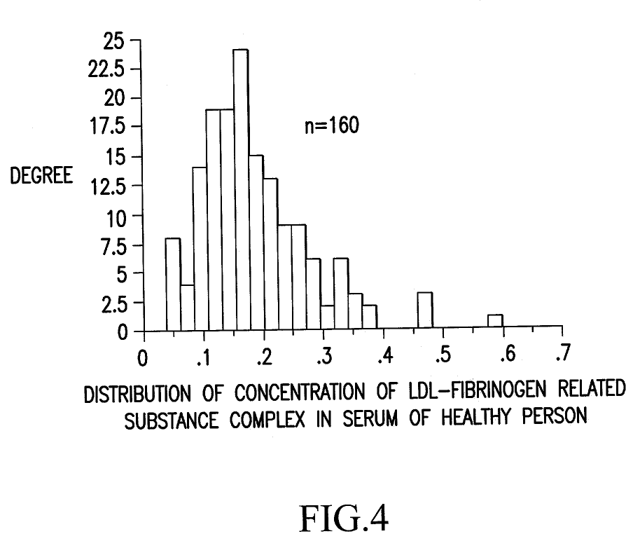

The concentration of an LDL-fibrinogen related substance in serum of a healthy person is as shown in FIG. 4.

(E-9) Amount of LDL-Fibrinogen Related Substance Complex in Serum of a Healthy Person, Diabetic Mellitus Patient and Multiple Risk Factor Syndrome Patient

The amounts of an LDL-fibrinogen related substance complex in a diabetic mellitus patient and multiple risk factor syndrome patient were significantly higher as compared with that of a healthy person.

(E-10) Comparison of Amounts of Oxidized LDL-α1 Antitrypsin, Oxidized LDL-Fibrinogen, Oxidized LDL-SAA, Oxidized LDL-CRP Complexes in the Serums of a Healthy Person and a Coronary Artery Disease Patient

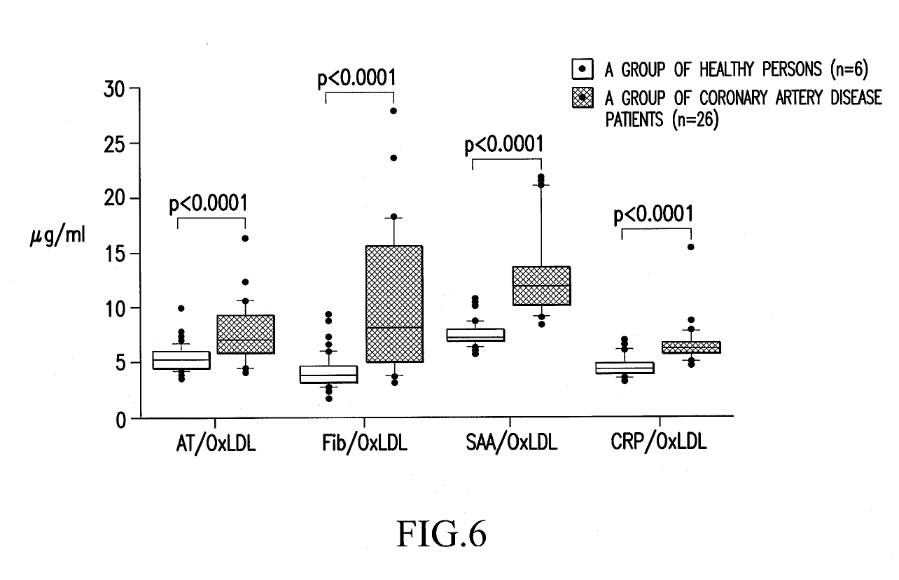

As shown in FIG. 6, the amounts of oxidized LDL-α1 antitrypsin, oxidized LDL-fibrinogen, oxidized LDL-SAA, oxidized LDL-CRP complexes in the serums of a group of coronary artery disease patients were significantly greater than those of healthy persons.

(E-11) Review of the Formation Mechanism of Oxidized LDL-(Serum Amyloid A1; SAA1) Complex

SAA is mostly present in HDL. As long as it exists in HDL, SAA is known to work against artery hardening. The present inventors have found that this anti-arteriosclerotic action takes effect as SAA in HDL molecules combines with LDL following the oxidization of LDL.

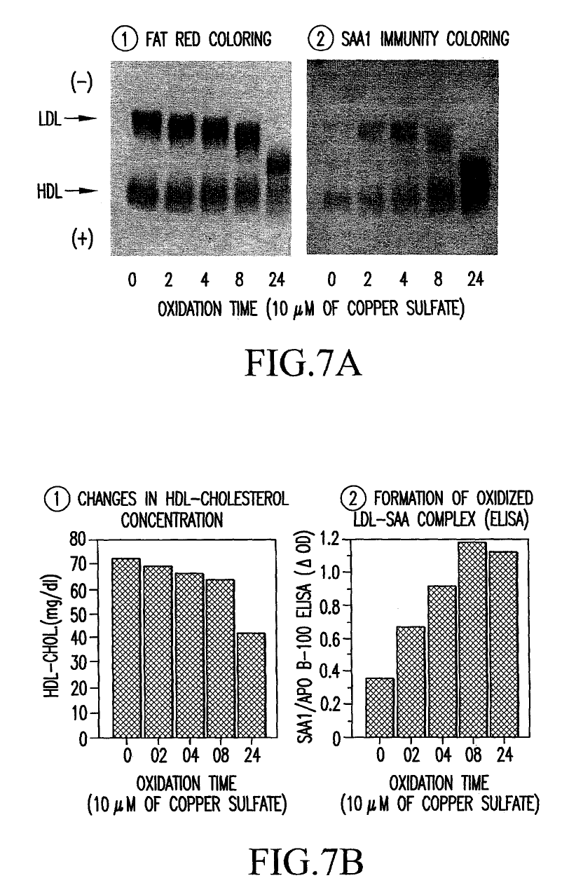

In other words, when equal amounts of native LDL and native HDL were mixed; 10 μAM of copper sulfate was added; and the mixture was left at 37° C., oxidized LDL-SAA complex was formed in accordance with the degree of oxidization (FIG. 7, A-\mathring{2}, B-\mathring{2}).

What is claimed is:

1. A method for arteriosclerosis diagnosis comprising the steps:
sampling blood drawn from a patient or an LDL fraction prepared from the said blood;
measuring quantitatively by an immunological measuring means a concentration of a complex present in the said sample comprising oxidized LDL and a marker protein selected from the group consisting of an acute phase reactant protein, blood coagulation-fibrinolytic related protein and a disinfectant substance produced by macrophages; and
diagnosing the onset and progress of arteriosclerosis by determining whether the said concentration is significantly higher than that of a group of healthy persons.

2. The method for arteriosclerosis diagnosis as recited in claim 1, wherein the acute phase reactant is a protein selected from the group consisting of a 1-antitrypsin, fibrinogen, fibronectin, lipoprotein (a), C-reactive protein (CRP), serum amyloid A (SAA), serum amyloid P component (SAP), α2-macroglobulin, α1-antichymotrypsin, α1-acidoglycoprotein and a complement component.

3. The method for arteriosclerosis diagnosis as recited in claim 1, wherein the blood coagulation fibrinolytic related protein is a protein selected from the group consisting of a tissue factor, plasminogen, prothrombin, thrombin, antithrombin 3 and a plasmin activator inhibitor 1.

4. The method for arteriosclerosis diagnosis as recited in claim 1, wherein the disinfectant substance produced by macrophages is a protein selected from the group consisting of myeloperoxyidase, lactroferrin, lysozyme and basic protein.

5. The method for arteriosclerosis diagnosis as recited in claim 1, wherein the immunological measuring means is a known means selected from the group consisting of an enzyme immunoassay, a latex flocculation method, an immunological emission spectrochemical analysis and an immunochromato method.

6. The method for arteriosclerosis diagnosis as recited in claim 1, wherein the immunological measuring means is a known means selected from the group consisting of an enzyme immunoassay, a latex flocculation method, an immunological emission spectrochemical analysis and an immunochromato method, characterized by the use of an antibody for detecting a marker protein forming a complex with oxidized LDL and/or an antibody for detecting ApoB100 present in oxidized LDL forming a complex with the said marker protein.

7. The method for arteriosclerosis diagnosis as recited in claim 1, wherein the immunological measuring means is a known means selected from the group consisting of an enzyme immunoassay, a latex flocculation method, an immunological emission spectrochemical analysis and an immunochromato method, characterized by the use of an antibody for detecting a marker protein forming a complex with oxidized LDL, as a primary antibody and an antibody for detecting ApoB100 present in oxidized LDL forming a complex with the said marker protein, as a secondary antibody.

8. The method for arteriosclerosis diagnosis as recited in claim 1, wherein the immunological measuring means is a known means selected from the group consisting of an enzyme immunoassay, a latex flocculation method, an immunological emission spectrochemical analysis and an immunochromato method, characterized by the use of a monoclonal antibody specifically recognizing a marker protein forming a complex with oxidized LDL, as a primary antibody and an antibody for detecting ApoB100 present in oxidized LDL forming a complex with the said marker protein, as a secondary antibody.

9. The method for arteriosclerosis diagnosis as recited in claim 1, wherein the immunological measuring means is a known means selected from the group consisting of an enzyme immunoassay, a latex flocculation method, an immunological emission spectrochemical analysis and an immunochromato method, characterized by the use of an antibody for detecting a marker protein forming a complex with oxidized LDL, as a primary antibody and a monoclonal antibody specifically recognizing ApoB100 present in oxidized LDL forming a complex with the said marker protein, as a secondary antibody.

10. The method for arteriosclerosis diagnosis as recited in claim 1, wherein the LDL fraction is obtained from a patient's blood by an ultra-centrifugal method or by reacting a dextran sulfate reagent.
